(19) United States
(12) Patent Application Publication
(10) Pub. No.: US 2025/0118445 A1
(43) Pub. Date: Apr. 10, 2025

(54) HEART FAILURE READMISSION

(71) Applicant: Cardiac Pacemakers, Inc., St. Paul, MN (US)

(72) Inventors: Jonathan Bennett Shute, Eagan, MN (US); Ramesh Wariar, Blaine, MN (US); Stephen B. Ruble, Lino Lakkes, MN (US); Sean Thomas Horan, Maplewood, MN (US); Brian Kwan, Mounds View, MN (US); Craig Stolen, New Brighton, MN (US)

(21) Appl. No.: 18/904,366

(22) Filed: Oct. 2, 2024

Related U.S. Application Data
(60) Provisional application No. 63/588,608, filed on Oct. 6, 2023.

(51) Int. Cl. G16H 50/30; A61B 5/00; A61B 5/0205; A61B 5/07; A61B 7/02; G16H 40/63

(52) U.S. Cl. CPC G16H 50/30; A61B 5/0205; A61B 5/076; A61B 5/686; A61B 5/7282; A61B 5/747; G16H 40/63; A61B 5/4836; A61B 7/023

(57) ABSTRACT

Systems and methods are disclosed to optimize resources of a medical device system, including to analyze physiologic information of a patient occurring over specific time periods relative to a prior heart failure event, to adjust, in response to a detected or received trigger event, a baseline value for the patient from a long-term baseline value to one of a shorter-term baseline value or a static value indicative of a baseline value at a prior time relative to the detected or received trigger event, to determine a readmission score for the patient based on the analyzed physiologic information, and to provide a control signal to control a mode or operation of the medical device system based on the readmission score to optimize resources of the medical device system.

### CLAIM OF PRIORITY

This application claims the benefit of U.S. Provisional Application No. 63/588,608, filed on Oct. 6, 2023, which is hereby incorporated by reference in its entirety.

### TECHNICAL FIELD

This document relates generally to medical devices and more particularly, but not by way of limitation, to systems and methods to determine a heart failure readmission metric for a patient.

### BACKGROUND

Heart failure (HF) is a reduction in the ability of the heart to deliver enough blood to meet bodily needs. Heart failure patients commonly have enlarged hearts with weakened cardiac muscles, resulting in reduced contractility and poor cardiac output. Signs of heart failure include pulmonary congestion, edema, difficulty breathing, etc. Heart failure is often a chronic condition, but can also occur suddenly, affecting the left, right, or both sides of the heart. Causes of heart failure include coronary artery disease, myocardial infarction, high blood pressure, atrial fibrillation, valvular heart disease, alcoholism, infection, cardiomyopathy, or one or more other conditions leading to a decreased pumping efficiency of the heart.

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition or identification of patients or groups of patients having elevated risk of future adverse events.

### SUMMARY

Systems and methods are disclosed analyze physiologic information of a patient occurring over specific time periods relative to a prior heart failure event, to adjust, in response to a detected or received trigger event, a baseline value for the patient from a long-term baseline value to one of a shorter-term baseline value or a static value indicative of a baseline value at a prior time relative to the detected or received trigger event, and to determine a readmission score for the patient based on the analyzed physiologic information, the readmission score for the patient indicative of a risk of subsequent readmission after treatment or discharge from hospitalization or treatment of the prior heart failure event.

An example of subject matter (e.g., a medical device system for improving heart failure readmission risk determination to optimize resources of the medical device system) may comprise means for receiving physiologic information of a patient means for analyzing the physiologic information occurring over specific time periods relative to a prior heart failure event with respect to a baseline value for the patient means for adjusting, in response to a detected or received trigger event, the baseline value for the patient from a long-term baseline value to one of a shorter-term baseline value or a static value indicative of a baseline value at a prior time relative to the detected or received trigger event means for determining a readmission score for the patient based on the analyzed physiologic information, the readmission score for the patient indicative of a risk of subsequent readmission after treatment or discharge from hospitalization or treatment of the prior heart failure event and means for providing a control signal to control a mode or operation of the medical device system based on the readmission score to optimize resources of the medical device system.

In an example, which may be combined with any one or more examples described herein, the means for receiving, analyzing, adjusting, determining, and providing comprise a signal receiver circuit configured to receive physiologic information of a patient and an assessment circuit configured to analyze the physiologic information occurring over specific time periods relative to a prior heart failure event with respect to a baseline value for the patient adjust, in response to a detected or received trigger event, the baseline value for the patient from a long-term baseline value to one of a shorter-term baseline value or a static value indicative of a baseline value at a prior time relative to the detected or received trigger event determine a readmission score for the patient based on the analyzed physiologic information, the readmission score for the patient indicative of a risk of subsequent readmission after treatment or discharge from hospitalization or treatment of the prior heart failure event and provide a control signal to control a mode or operation of the medical device system based on the readmission score to optimize resources of the medical device system.

An example of subject matter (e.g., a medical device system for improving heart failure readmission risk determination to optimize resources of the medical device system) may comprise a signal receiver circuit configured to receive physiologic information of a patient and an assessment circuit configured to analyze the physiologic information occurring over specific time periods relative to a prior heart failure event with respect to a baseline value for the patient adjust, in response to a detected or received trigger event, the baseline value for the patient from a long-term baseline value to one of a shorter-term baseline value or a static value indicative of a baseline value at a prior time relative to the detected or received trigger event determine a readmission score for the patient based on the analyzed physiologic information, the readmission score for the patient indicative of a risk of subsequent readmission after treatment or discharge from hospitalization or treatment of the prior heart failure event and provide a control signal to control a mode or operation of the medical device system based on the readmission score to optimize resources of the medical device system.

An example of subject matter (e.g., a method for improving heart failure readmission risk determination to optimize resources of a medical device system) may comprise receiving, using a signal receiver circuit, physiologic information of a patient analyzing, using an assessment circuit, the physiologic information occurring over specific time periods relative to a prior heart failure event with respect to a baseline value for the patient adjusting, using the assessment circuit, in response to a detected or received trigger event, the baseline value for the patient from a long-term baseline value to one of a shorter-term baseline value or a static value indicative of a baseline value at a prior time relative to the detected or received trigger event determining, using the assessment circuit, a readmission score for the patient based on the analyzed physiologic information, the readmission score for the patient indicative of a risk of subsequent readmission after treatment or discharge from hospitalization or treatment of the prior heart failure event and providing, using the assessment circuit, a control signal to control a mode or operation of the medical device system based on the readmission score to optimize resources of the medical device system.

In an example, which may be combined with any one or more examples described herein, to determine or determining the readmission score includes to determine or determining the readmission score with respect to the adjusted baseline value for the patient.

In an example, which may be combined with any one or more examples described herein, to provide or providing the control signal based on the readmission score includes in response or responsive to, such as triggered by, the determined readmission score exceeding the adjusted baseline value by a threshold.

In an example, which may be combined with any one or more examples described herein, to determine or determining the readmission score for the patient includes as a composite function of the analyzed physiologic information, including two or more of: S1 heart sound information, S3 heart sound information, thoracic impedance, activity information, respiration information, and heart rate.

In an example, which may be combined with any one or more examples described herein, the signal receiver circuit or the assessment circuit is configured to receive, or the method includes receiving, an indication of patient discharge from hospitalization or treatment of the prior heart failure event, wherein to determine or determining the readmission score for the patient includes to determine or determining an indication that the patient is likely to experience readmission or an indication that the prior heart failure event and discharge is likely to result in readmission within a specific time period after the received indication of patient discharge from hospitalization or treatment of the prior heart failure event.

In an example, which may be combined with any one or more examples described herein, the readmission score is indicative of a future heart failure event.

In an example, which may be combined with any one or more examples described herein, the assessment circuit is configured to determine or the method includes determining the baseline value for the patient, including to determine or determining the long-term baseline value as a function of 30 or more days of preceding physiologic information of the patient and determine the shorter-term baseline value as a function of a time period of physiologic information of the patient between 3 and 10 days of preceding physiologic information of the patient, wherein to adjust or adjusting the baseline value for the patient includes to adjust the baseline value for the patient from the long-term baseline to the shorter-term baseline value.

In an example, which may be combined with any one or more examples described herein, the assessment circuit is configured to determine or the method includes determining the baseline value for the patient, including to determine or determining the long-term baseline value as a function of 30 or more days of preceding physiologic information of the patient and determine the static value indicative of the baseline value at a prior time relative to the detected or received trigger event, the prior time including at least 30 days prior to the detected or received trigger event, wherein to adjust or adjusting the baseline value for the patient includes to adjust the baseline value for the patient from the long-term baseline to the static value.

In an example, which may be combined with any one or more examples described herein, the assessment circuit is configured to determine or the method includes determining the baseline value as a function of the long-term baseline value, the shorter-term baseline value, and the static value over a transition period until the determined readmission score or the long-term baseline meets the static value.

In an example, which may be combined with any one or more examples described herein, to analyze or analyzing the physiologic information includes to analyze or analyzing S3 heart sound information over first and second time periods and to determine the readmission score for the patient as function of a difference in the S3 heart sound information over the first and second time periods, wherein the first time period is before the prior heart failure event and the second time period is after discharge from hospitalization or treatment of the prior heart failure event.

In an example, which may be combined with any one or more examples described herein, the first time period is between −25 days and −5 days before the prior heart failure event and the second time period is between 0 days and 30 days after discharge from hospitalization or treatment of the prior heart failure event.

In an example, which may be combined with any one or more examples described herein, to determine or determining the readmission score includes as a function of a first composite function of the analyzed physiologic information over a first time period before the prior heart failure event, the physiologic information including two or more of: S1 heart sound information, S3 heart sound information, thoracic impedance, activity information, respiration information, and heart rate information a second composite function of S3 heart sound information and S1 heart sound information over a time period before the prior heart failure event and an integrated value of the first composite function over a second time period after discharge from hospitalization or treatment of the prior heart failure event.

In an example, which may be combined with any one or more examples described herein, the trigger event is one or more of a received request to adjust the respective baseline value, a detected in-alert state, or a received or detected indication of a first heart failure event or hospitalization or treatment of the first heart failure event of the patient, wherein the first heart failure event is the prior heart failure event.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-5 illustrate example physiologic values for a plurality of patients leading up to admission or treatment for a heart failure event.

FIG. 6 illustrates example baseline determinations, including a prior 30-day baseline, a shorter 5-day baseline, and a pre-alert baseline.

FIG. 7 illustrated an example method.

FIG. 8 illustrates an example medical device system.

FIG. 9 illustrates an example patient management system.

FIG. 10 illustrates an example implantable medical device system.

FIG. 11 illustrates example implantable medical devices.

FIG. 12 illustrates an example remote patient management system.

FIG. 13 illustrates an example machine upon which any one or more of the techniques discussed herein may perform.

### DETAILED DESCRIPTION

Ambulatory medical devices are devices configured to be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) devices configured to monitor or provide stimulation to the patient and, in certain examples, one or more circuits to detect one or more physiologic events using the sensed physiological information or transmit or receive sensed physiologic information or detected physiologic events to one or more remote devices. Frequent patient monitoring can provide early detection of worsening patient condition or identification of patients or groups of patients having elevated risk of future adverse events.

Ambulatory medical devices can provide different monitoring, storage, communication, or therapy using different modes, however, with different power and resource requirements and varying effectiveness for different patients. For example, a variety of therapy modalities are available to patients, but not all patients receive the optimal medical device, therapy mode, or therapy parameter settings at first programming. One common reason for suboptimal ambulatory medical device programming is detection or determination of different events, conditions, or indications. Optimal programming depends on, among other things, accurate detection and determination of different events, conditions, or indications. Desired clinical outcomes can include, among others, cardiac capture, selection of pacing or sensing vectors, pacing mode, resource usage associated with communication or transmission of physiologic data from the ambulatory medical device or storage of physiologic data, etc.

Identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, reduce healthcare costs, improve patient management, and direct limited healthcare resources to patients most in need, for example, to optimize resources of a medical device system comprising the ambulatory medical device.

The present inventors have recognized, among other things, systems and methods to determine a heart failure readmission risk for a patient, such as for predicting a risk of readmission or rehospitalization for a patient within one or more long-term future time periods (e.g., greater than 30 days, 90 days, etc.) following a detected or received heart event, including a hospitalization event or a received indication of discharge from a hospitalization event, etc. The systems and methods disclosed herein can include a single- or multi-sensor-based readmission risk metric (e.g., a readmission score), automatically determined and updated at one or more regular or adjustable intervals. In certain examples, the readmission risk metric can predict a patient-specific risk of readmission (or rehospitalization) at different time intervals (e.g., within the next 7 days, 14 days, 30 days, 90 days, etc.) or as a combination of a set of risks at several points in the future. Information about the readmission risk metric can be used to trigger changes in device operation, transition between different operating modes, to cause alerts or notifications to be provided to an external device or process, or trigger or cause one or more other device adjustments to improve the performance of one or more devices associated with the patient to optimize resources of a medical device system comprising the one or more devices. For example, triggering a change in operation, such as changing a monitoring mode to increase resources with respect to sensing, storing, computing, or transmitting physiologic information or different metrics responsive to a determined increase or indication of readmission risk (e.g., the readmission risk metric above a threshold, etc.), while reducing or limiting such resource allocation during normal monitoring periods, can improve device efficiency and optimize efficient use of medica devices including ambulatory or implantable medical devices or sensors having a limited power supply or resources available, thereby optimizing efficient use of such devices with the patient.

The single- or multi-sensor-based readmission risk metric can be determined, in certain examples, using different physiologic information of the patient or various combinations thereof, such as heart sound information (e.g., S3/S1, S3, S1, etc.), respiration information (respiratory rate (RR), tidal volume (TV), rapid shallow breathing index (RSBI), etc.), activity information, impedance information, temperature information, posture information, arrhythmia information, electrolyte information, oxygen saturation information, a composite measurement, or combinations thereof.

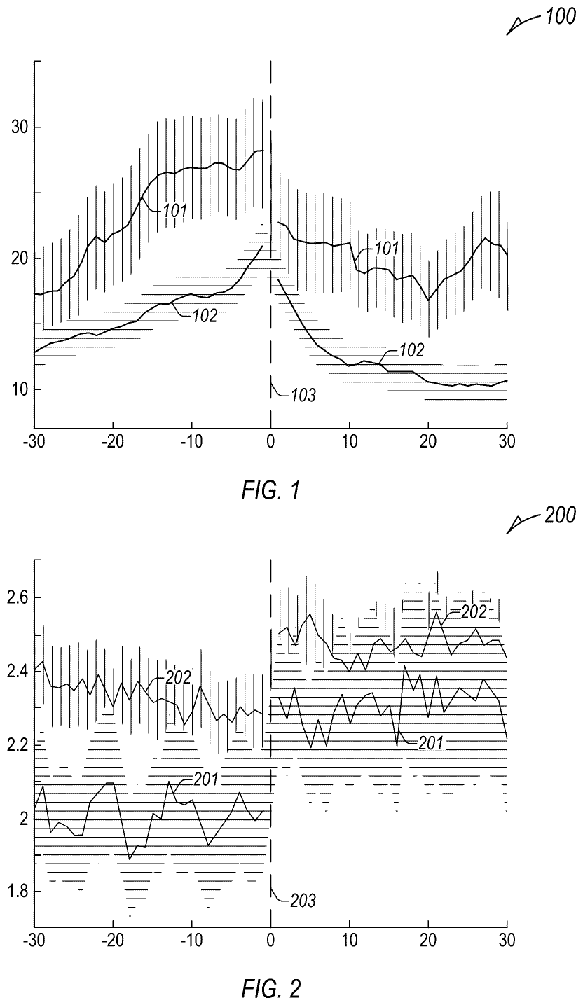

The composite measurement can include a HeartLogic™ index, a HeartLogic™ in-alert time, or one or more other composite measurements or measures thereof. The HeartLogic™ index is a composite health index determined as a function of different physiologic information from one or more ambulatory sensors, including S1 and S3 heart sounds, thoracic impedance, activity information, respiration information, and nighttime heart rate (nHR), and is indicative of a heart failure status, a risk of a future heart failure event, or a worsening of the heart failure status or risk of heart failure event in the patient over time. The HeartLogic™ in-alert time is a measure of time that the HeartLogic™ index is above an alert threshold (e.g., a nominal value of 16, etc., but programmable, such as by a clinician to one or more other higher or lower values). In certain examples, where the HeartLogic™ index value output can include raw values, such as illustrated along the y-axis of FIGS. 1-6, the information used to determine the HeartLogic™ index value can include relative values, such as values with respect to one or more determined patient baselines (e.g., a difference between or ration of a daily or short-term (e.g., 3 days, 7 days, 14 days, etc.) value and a patient baseline or long-term value determined over a relatively longer period of time (e.g., 30 days or longer, etc.), etc.).

In certain examples, the HeartLogic™ index can be determined using different combinations or weightings of physiologic information, including one or more of S1 and S3 heart sounds, thoracic impedance, activity information, rapid shallow breathing index (RSBI), respiratory rate, and nighttime heart rate (nHR), including, in certain examples, changes in one or more measures with respect to a long-term or baseline information from the patient. In certain examples, the different combinations or weightings of the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.).

For example, if the risk stratifier is low, or below a first threshold, the HeartLogic™ index can be determined using a first combination of physiologic information. If the risk stratifier is high, or above a second threshold, the HeartLogic™ index can be determined using the first combination of physiologic information and a second combination of physiologic information, including additional information than included in the first combination. If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

The present inventors have recognized, among other things, that a risk of readmission following discharge associated with treatment of a heart failure event can be determined using the determined HeartLogic™ index or components thereof, over a time period leading up to a heart failure event, such as a detected or received indication of hospitalization, or after discharge from or associated with treatment of the detected or received indication of hospitalization. For example, as illustrated in FIGS. 1-6, trends in the determined HeartLogic™ index are significantly different for patients with readmissions within 30 or 90 days after discharge associated with a heart failure event in contrast to patients without readmissions within 30 or 90 days. The determined HeartLogic™ index values leading up to the detected event, or in other examples, S1 or S3 heart sound information in combination with or separate from the determined HeartLogic™ index can be used to determine a risk indication for a future heart failure event or a likelihood of readmission in a time period after discharge or treatment associated with a heart failure event.

Further, the present inventors have recognized that one or more determined baselines or long-term values can be adjusted or changed following a heart failure event, such as to more accurately track patient progress or improvement following discharge, reducing the impact of changing signals prior to a heart failure event on post-event monitoring, and more accurately determining and tracking patient progress and stability following discharge. If the determined baseline includes changing values leading up to the heart failure event that required treatment or hospitalization, improvements in the HeartLogic™ index following discharge can be overvalued, or rising values or gradual increases following discharge can be undervalued due to the prior higher values. Whereas the existing HeartLogic™ index can be determined using combinations of daily or short-term values relative to a baseline or long-term (e.g., 30 days or longer, etc.) value and subsequent comparison to a threshold, the present inventors have determined that a shorter baseline (e.g., 5 days, 7 days, 10 days, etc., substantially shorter period than the existing long-term period) can be advantageous following discharge associated with treatment of a heart failure event, more accurately reflecting changing patient status following discharge until a new long-term baseline can be established without consideration of the changing values leading up to the heart failure event that required treatment or hospitalization. In addition or alternatively, a pre-alert baseline, such as the baseline from 30 days prior to the heart failure event, can be carried over until changing values leading up to the heart failure event that required treatment or hospitalization have returned to the previous or a new baseline value. Once the HeartLogic™ index values approach a new baseline value following the heart failure event (e.g., 30 days post-discharge or longer, etc.), the new baseline can replace the pre-alert or shorter baseline, or a smoothing period (e.g., a week, etc.) between the pre-alert or shorter baseline and the new baseline can be implemented where a function of the different baselines can ease the transition between the different values.

In other examples, an index event recovery score can be determined based on changes in the determined HeartLogic™ index values, or in other examples, S1 or S3 heart sound information in combination with or separate from the determined HeartLogic™ index, in one or more time periods (e.g., 7 days post discharge, 14 days post discharge, 21 days post discharge, or combinations thereof, in certain examples using one or more adjusted baselines as described herein) following discharge associated with a heart failure event. For example, changes indicative of continued improvement or stabilization can provide higher index event recovery scores than changes indicative of instability or decline. In addition, determination of the index event recovery score can use the pre-alert or shorter baseline described above or combinations thereof to provide more accurate recovery indications than the existing HeartLogic™ index value at commensurate points.

In certain examples, the single- or multi-sensor-based readmission risk metric can be a device-based metric, without input of clinical information about the patient, such as clinician diagnosis or determination of risk, patient history, patient age, comorbidities, prior hospitalization, type of implanted device, etc. In other examples, the mortality risk metric can be a combination of a device-based and clinical-based readmission risk metric, including or considering clinical information about the patient, such as clinician diagnosis or determination of risk, patient history, patient age, comorbidities, prior hospitalization, type of implanted device, patient reported outcomes (related to symptoms or quality of life), etc. In certain examples, patient history can include a previous record of diagnosis, intervention, or treatment for one or more conditions, including chronic obstructive pulmonary disease (COPD), chronic kidney disease (CKD), anemia, heart failure, etc. In an example, separate determinations can be made for different combinations of clinical information. Table 1, below, illustrates additional clinical risk factors and determined univariate odds ratio (OR), 95% confidence interval (CI), and p-value from logistic regression (P-value) for a group of predictors. Values substantially higher or lower than 1 are significant predictors, including, expectedly, diuretics, but not expectedly, race and depression.

TABLE 1
Clinical Risk Factors (30-day Primary HF Readmission)

| Predictor | OR | 95% CI | P-value |
|---|---|---|---|
| NYHA Class III/IV | 2.1 | 0.64-7.01 | 0.22 |
| Age >65 | 1.1 | 0.33-3.43 | 0.92 |
| Sex male | 1.4 | 0.40-4.76 | 0.62 |
| Race white | 0.3 | 0.09-0.91 | 0.033 |
| Prior heart failure hospitalization | 1 | 0.32-3.03 | 0.99 |
| Myocardial infarction | 1.8 | 0.57-5.38 | 0.33 |
| Coronary artery bypass graft | 1.5 | 0.45-4.86 | 0.52 |
| COPD | 0.3 | 0.03-2.35 | 0.24 |
| Cerebrovascular disease | 1.5 | 0.35-6.00 | 0.6 |
| Atrial fibrillation | 0.3 | 0.09-1.29 | 0.11 |
| Ischemic heart disease | 1.2 | 0.40-3.85 | 0.7 |
| Hypertension | 1 | 0.25-4.00 | 1 |
| Diuretics | 3.8 | 0.46-31.09 | 0.21 |
| Depression | 6.5 | 1.78-23.60 | 0.005 |

FIG. 1 illustrates example relationship 100 of HeartLogic™ index values (y axis) for a plurality of patients, including a yes-readmission group 101 (20 patients) and a no-readmission group 102 (83 patients), for a pre-event time period, 30 days prior to (−30) and leading up to a heart failure event 103 at day 0 and treatment or hospitalization, after day 0 and including a post-discharge time period, 30 days after discharge or treatment associated with the heart failure event 103. The time period for treatment or hospitalization can be represented day 0, in certain examples collapsed into a single time period, although frequently variable among different patients and clinicians. Although variable within and among the different groups of patients (e.g., between 2 and 14 days), the length of treatment time between admission and discharge during the time period for treatment was surprisingly not a statistical indicator for either of the yes-readmission group 101 or the no-readmission group 102.

As illustrated in FIG. 1, the yes-readmission group 101 starts with a substantially higher HeartLogic™ index value at −30 days than the no-readmission group 102 (about 17.5 versus 13), has a faster rise between days −30 and −10 (to 26 versus 16, a nearly 50% increase versus a 23% increase), is in an in-alert state (a HeartLogic™ index value greater than a threshold, such as 16, etc.) longer, and maintains a higher value between days −15 and 0 (over 26 for a full 15 days) than the no-readmission group 102. For example, if the alert threshold is 16, whereas the no-readmission group 102 is in an in-alert state greater than the threshold less than 14 days prior to the heart failure event 103, the yes-readmission group 101 is in the in-alert state more than 30 days prior to the heart failure event 103. The HeartLogic™ index value illustrated in FIG. 1 can generally be used as a predictor of readmission, even before the first heart failure event 103. The present inventors have recognized, in particular, that 30 days of continuous high alert or in-alert time (e.g., having a HeartLogic™ value greater than or equal to 16 or one or more other thresholds indicative of a high index value or exceeding an alert level) before a heart failure event or hospitalization is a strong predictor of readmission risk following discharge or treatment of the heart failure event or hospitalization. Information during and following treatment can confirm or further specify the likelihood of readmission, but the prediction can be determined prior to the first heart failure event 103.

In addition, the relative decrease during treatment or hospitalization (day 0) is greater for the yes-readmission group 101 (falling from 27 to 22, an 18.5% decrease) than the no-readmission group 102 (falling from 21 to 18, a 14% decrease). The yes-readmission group 101 is discharged at approximately the HeartLogic™ index value that the no-readmission group 102 is admitted. Post discharge, the HeartLogic™ index value of the yes-readmission group 101 does not drop quickly or smoothly as the no-readmission group 102, and further rises between days 20 and 30 post-discharge, in contrast to the no-readmission group 102.

FIG. 2 illustrates example relationship 200 of S1 heart sound values for a plurality of patients, including a yes-readmission group 201 and a no-readmission group 202, for a pre-event time period, 30 days prior to and leading up to a heart failure event 203 and treatment or hospitalization, represented by day 0, and a post-discharge time period, 30 days after discharge or treatment associated with the heart failure event 203.

As illustrated in FIG. 2, the yes-readmission group 201 starts with a substantially lower S1 value at −30 days than the no-readmission group 202 (about 2 versus 2.4), and is much more volatile between days −30 and 0, although with a substantially even slope versus the generally decreasing slope of the no-readmission group 202 between days −30 and 0. In addition, the relative increase of S1 value during treatment or hospitalization (day 0) is greater for the yes-readmission group 201 (rising to 2.3, a 15% increase) than the no-readmission group 202 (rising to 2.5, an 8.7% increase). The S1 heart sound values, in particular, can be used to stratify the different readmission and no-readmission groups 201, 202, such as by comparing the S1 value at the heart failure event 203 or during the 30 days preceding the heart failure event 203 to a threshold, such as 2.2, etc.

Like the HeartLogic™ index values in FIG. 1, the yes-readmission group 201 is discharged at approximately the S1 value that the no-readmission group 202 is admitted. Post discharge, the S1 values of the yes-readmission group 201 are generally more variable and generally slightly increasing in contrast to the relatively flat no-readmission group 202.

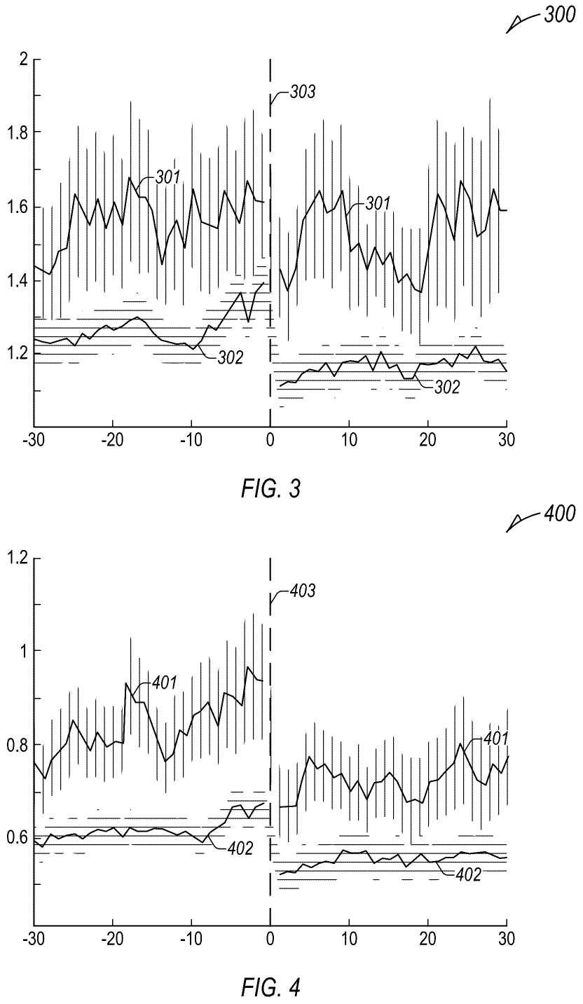

FIG. 3 illustrates example relationship 300 of S3 heart sound values for a plurality of patients, including a yes-readmission group 301 and a no-readmission group 302, for a pre-event time period, 30 days prior to and leading up to a heart failure event 303 and treatment or hospitalization, represented by day 0, and a post-discharge time period, 30 days after discharge or treatment associated with the heart failure event 303.

As illustrated in FIG. 3, the yes-readmission group 301 starts with a substantially higher S3 value at −30 days than the no-readmission group 302 (about 1.45 versus 1.25) and is much more volatile between days −30 and 0. Like the HeartLogic™ index values in FIG. 1 and the S1 values in FIG. 2, the yes-readmission group 301 is discharged at approximately the S3 value that the no-readmission group 302 is admitted. Post discharge, the S3 values of the yes-readmission group 301 are generally more variable and unstable and higher than the relatively stable no-readmission group 302. The S3 values can be used to stratify and predict the different readmission and no-readmission groups 301, 302, for example, setting threshold values pre- and post-discharge particularly due to separation of S3 values between days −25 and −5 and days 0 to 30 post-discharge, etc.

FIG. 4 illustrates example relationship 400 of S3/S1 heart sound values (S3 value normalized by S1 value) for a plurality of patients, including a yes-readmission group 401 and a no-readmission group 402, for a pre-event time period, 30 days prior to and leading up to a heart failure event 403 and treatment or hospitalization, represented by day 0, and a post-discharge time period, 30 days after discharge or treatment associated with the heart failure event 403.

As illustrated in FIG. 4, the yes-readmission group 401 starts with a substantially higher S3/S1 value at −30 days than the no-readmission group 402 (about 0.75 versus 0.6) and is much more volatile between days −30 and 0 and generally increasing in contrast to the no-readmission group 402, which is generally stable and does not increase until day −10. Like the HeartLogic™ index values in FIG. 1, the S1 values in FIG. 2, and the S3 values in FIG. 3, the yes-readmission group 401 is discharged at approximately the S3/S1 value that the no-readmission group 402 is admitted. Post discharge, the S3/S1 values of the yes-readmission group 401 are generally more variable and unstable and higher than the relatively stable no-readmission group 402. The S3/S1 values can be used to stratify and predict the different readmission and no-readmission groups 401, 402, for example, setting threshold values pre- and post-discharge (e.g., 0.7 pre and 0.65 post, etc.), particularly due to separation of S3/S1 values throughout the different time periods, etc.

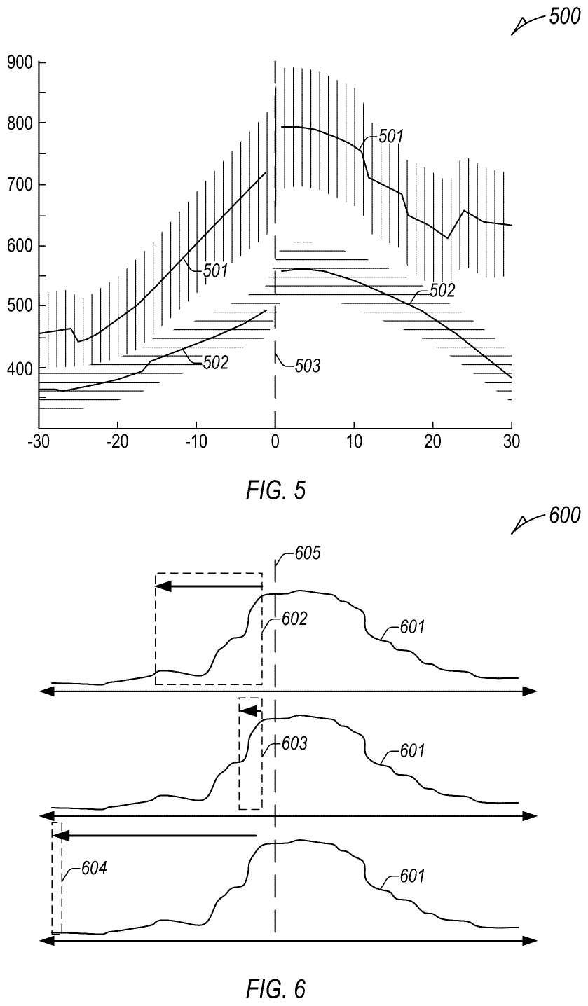

FIG. 5 illustrates example relationship 500 of integrated HeartLogic™ index values for a plurality of patients, including a yes-readmission group 501 and a no-readmission group 502, for a pre-event time period, 30 days prior to and leading up to a heart failure event 503 and treatment or hospitalization, represented by day 0, and a post-discharge time period, 30 days after discharge or treatment associated with the heart failure event 503. The integrated HeartLogic™ index values at each day represent the integrated 30 previous days of HeartLogic™ index values (e.g., daily values, one per day).

As illustrated in FIG. 5, the readmission group 501 starts with a higher integrated HeartLogic™ index value at −30 days than the no-readmission group 402 (about 460 versus 375) and increases at a substantially higher rate between days −20 and 0 (about 50% versus 33%). There is substantial separation in the integrated HeartLogic™ index values between the readmission and no-readmission groups 501, 502 at day 0 (700 versus 500). In contrast to the values in FIGS. 1-4, the readmission group 501 is not discharged at approximately the integrated HeartLogic™ index value that the no-readmission group 502 is admitted. Both values increase during treatment or hospitalization at day 0, with the increase for the readmission group 501 only growing. Post discharge, the integrated HeartLogic™ index values of the readmission group 501 are generally more variable and unstable and higher than the relatively declining no-readmission group 502, with clear separation between the groups. Accordingly, the integrated HeartLogic™ index values can be used to stratify and predict the different readmission and no-readmission groups 501, 502, for example, setting threshold values pre- and post-discharge (e.g., 600, etc.), particularly due to separation of such values throughout the different time periods, the difference in increase prior to day 0, etc.

FIG. 6 illustrates example relationship 600 of HeartLogic™ index values 601 for a patient and different baseline determinations, including an existing prior 30-day baseline 602, a shorter 5-day baseline 603, and a pre-alert baseline 604, such as from 30 days prior to a heart failure event 605 at day 0. In contrast to FIGS. 1-5, the time period of treatment or hospitalization is not collapsed in FIG. 6, and the elevated HeartLogic™ index values 601 are shown during treatment, such as hospitalization, where, for example, one or more medications such as diuretics, etc., can be applied to treat the heart failure event 605.

In certain examples, a baseline HeartLogic™ index values can be contaminated by rising HeartLogic™ index values preceding and leading up to a heart failure event. Changing the alert window can improve HeartLogic™ index value sensitivity post alert/hospitalization. For example, the existing 30-day baseline HeartLogic™ index value can be reduced to a shorter time period, such as T-X day baseline HeartLogic™ index values (e.g., a shorter-term baseline value), where X is substantially smaller than 30 (e.g., 5 days, etc.) to increase sensitivity to changes following discharge or post-alert. In other examples, the existing 30-day baseline HeartLogic™ index value can be held to a value prior to a detected in-alert state, or at a time interval before the detected heart failure event, such as a T-Y pre-alert baseline HeartLogic™ index value, where “Y” is 30 days prior to the detected event and the baseline is held at that value until the new baseline approaches that T-Y pre-alert baseline at the time of the detected event, etc., to reflect a patient current status versus a pre-hospitalization status.

The different baselines, such as the T-X or T-Y pre-baselines described herein, can effectively increase onset and reset thresholds for alert state determination by reducing impact of improving patient condition during hospitalization, illustrated in FIGS. 1-5, effectively improving determination of current patient status or condition with respect to determined risks of rehospitalization or future heart failure events. In other examples, the alert thresholds themselves, or reset thresholds once in an alert state, such as to account for short-term improvements in patient condition, can be adjusted in response to a detected or received indication of a heart failure event or associated hospitalization or treatment, for example, until a previous baseline is attained.

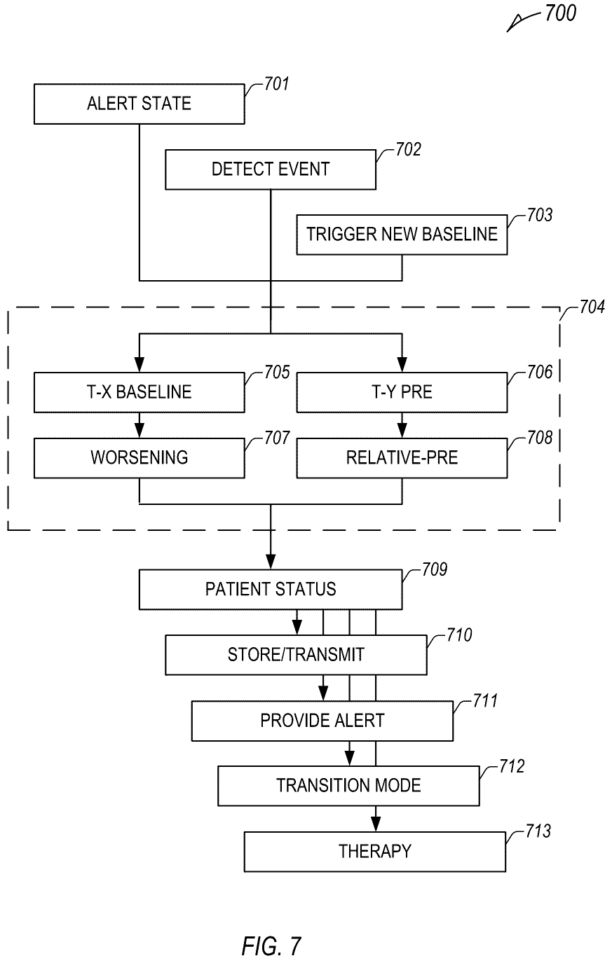

FIG. 7 illustrates an example method 700 including selecting or determining a new patient baseline for determination of the HeartLogic™ index value or one or more other composite values (e.g., a readmission score) after a heart failure event. In an example, physiologic information of a patient can be received, such as by a signal receiver circuit or one or more other circuits or components. The physiologic information can be analyzed, such as by an assessment circuit of an ambulatory medical device or one or more other circuits or components, such as of an external system, etc. In certain examples, physiologic information occurring over one or more specific time periods, such as relative to an alert state, a prior heart failure event, or one or more other triggers or events, can be analyzed, such as with respect to a baseline value for a patient, one or more thresholds, etc. In an example, analyzing the physiologic information can include measuring one or more parameters or values of or related to the physiologic information, or monitoring changing values or information over time or otherwise determining one or more other composite measures, scores, or indications.

At step 701, an alert state, such as an in-alert state or a HeartLogic™ index value above an alert threshold, can be detected, such as using the assessment circuit or one or more other circuits or components, such as described herein. In other examples, the alert state can be received, such as by the signal receiver circuit or one or more other circuits.

At step 702, an event, such as a heart failure event, hospitalization, or treatment, can be detected using one or more physiologic sensors, for example, detecting information indicative of treatment or a location of treatment, etc., or received, such as from a clinician, a hospital, or one or more systems associated with the patient indicative of treatment or hospitalization, including admission, a detected location, or proximity of one or more specific external system components, such as an external programmer, etc.

At step 703, a new baseline can be triggered, such as by a clinician or one or more other users. In certain examples, the new baseline can be triggered separately from or alternatively in response to one or more of the detected alert state or the detected event at steps 701, 702, etc.

At step 704, a new baseline window can be selected, such as in response to one or more of the detected alert state, the detected event, or the triggered new baseline at steps 701, 702, 703. Step 704 can optionally include one or both of a T-X baseline at step 705 or a T-Y pre-alert (T-Y PRE) baseline at step 706. The T-X baseline can include a relatively shorter baseline, where X is equal to 5 days or one or more shorter intervals, smaller than the existing 30-day baseline (e.g., 3 days, 5 days, 7 days, 10 days, etc.). The T-Y pre-alert baseline can include a pre-alert baseline, where Y is a number of days, such as 30 days, prior to the heart failure event held. The T-Y pre-alert can be held at that past baseline value for a time period (e.g., 30 days) after treatment or discharge associated with a heart failure event. In contrast, the T-X baseline can be a rolling baseline indicative of short-term changes of the patient (e.g., improving, stable, worsening, etc.). At step 707, a worsening indicator can be determined using a daily value or a combination of daily values relative to the T-X baseline, indicative of a short-term status (e.g., improvement or worsening) of the patient. At step 708, a relative pre-event status can be determined using a daily value or a combination of daily values relative to the T-Y pre-alert baseline, indicative of a relative status of the patient in contrast to a pre-heart failure event baseline of the patient.

In certain examples, at step 704, the new baseline window can be determined using the T-X baseline determined at step 705, the T-Y pre-alert baseline determined at step 706, or combinations thereof, such as a function of the one or both, and in certain examples, additionally as a function of the existing 30-day baseline otherwise described herein. For example, a transition period can blend a difference between the different baselines as time proceeds away from discharge, or day 0 as described herein.

At step 709, a patient status can be determined, such as a readmission score or one or more other determinations of readmission risk, such as the worsening indicator determined at step 707, the relative pre-event status determined at step 708, or combinations thereof. In an example, the function can additionally include a difference between the different baselines described herein, taking into account relative increases or decreases in baseline values in addition to the daily value or combination of daily values. For example, the function can include weighted combinations of one or more determinations relative to one or more other determinations to provide a score or indications of relative changes for the patient indicative of patient status (e.g., improvement or worsening, etc.), as well as determinations of new or adjusted baselines, and indications of heart failure readmission metrics or determined likelihoods of readmission (e.g., indicative of the likelihood that the patient will experience readmission) following discharge or treatment associated with a heart failure event.

Contractility and third heart sound (S1 and S3 respectively) show long term stratification power for readmissions. A low contractility and high third heart sound are strong risk factors. The determined patient status can indicate to users or clinicians, before a heart failure event, that the patient is potentially at a higher risk of a heart failure event or readmission for a subsequent heart failure event and should be monitored more closely, such as for alternative therapies, etc. For example, during treatment, and before discharge, a readmission score can be determined and provided or reported to the clinician that indicates likelihood of this hospitalization having a readmission. The readmission score can be determined, such as using the assessment circuit, as a composite of S3/S1, HeartLogic™ index values, and integrated HeartLogic™ index values. For example, the readmission score can be determined as a composite function of HeartLogic™ index values from before hospitalization or alert, S3/S1 from before hospitalization or alert, and integrated HeartLogic™ index values after discharge (e.g., such as the previous 30 days of integrated values, such as described and illustrated in FIG. 5, etc.).

For example, odds of readmission, comparing in alert vs out of alert can include values as follows: (1) if in alert 14 days prior to the index admission: 3.05 [1.02 (5%), 9.10 (95%)] times higher odds of readmission for Heart Failure in 30 days; 2.25 [0.84, 6.03] times higher odds for all-cause readmission in 30 days; (2) if in alert 7 days post discharge: 3.01 [1.29, 7.01] times higher odds of readmission for Heart Failure in 90 days; (3) if in alert 14 days post discharge: 2.67 [1.16, 6.14] times higher odds of readmission for Heart Failure in 90 days.

At step 710, determinations, indications, scores, or physiologic information from monitoring windows associated therewith, can be stored, such as using the assessment circuit, and transmitted, by control of the assessment circuit or using one or more communication circuits, etc., such as to one or more additional processes or components, such as an output circuit (e.g., a display, a controller for a display, etc.). At step 711, an alert can be optionally provided, such as by the assessment circuit, for example, if the determinations, indications, scores, or one or more baselines are available for review or transmission, if one or more of the determinations, indications, scores, or one or more baselines exceed a threshold, etc. In an example, an output can be provided of the determinations, indications, scores, or one or more baselines to a user interface for display to a user or to another circuit to control or adjust a process or a function of an implantable or ambulatory medical device.

At step 712, one or more modes or functions of the assessment circuit or an implantable or ambulatory medical device can be optionally adjusted based on one or more of the determinations, indications, scores, or one or more baselines. For example, if one or more the determinations, indications, scores, or one or more baselines exceeds one or more threshold or values, one or more hardware limitations can be adjusted, such as to, among others: record more or less physiologic information of the patient; increase communication frequency between the implantable or ambulatory medical device and an external device (e.g., remote device, programmer, etc.), such as to increase the frequency of patient monitoring, etc.; switch to a different or more power or resource intensive monitoring algorithm; etc.

At step 713, one or more therapies can be optionally provided or adjusted based on the determinations, indications, scores, or one or more baselines, etc., such as described herein.

Implantable or ambulatory medical devices can include or be configured to receive cardiac electrical information from one or more electrodes or sensors located within, on, or proximate to a heart, such as coupled to a lead and located in one or more chambers of the heart or within the vasculature of the heart near one or more chambers. Implantable or ambulatory medical devices can additionally include or be configured to receive mechanical acceleration information from one or more accelerometer sensors to determine and monitor patient acceleration information, such as cardiac acceleration or vibration information associated with blood flow or movement in the heart or patient vasculature (e.g., heart sounds, cardiac wall motion, etc.), patient physical activity or position information (e.g., patient posture, activity, steps, etc.), respiration information (e.g., respiration rate, volume, phase, breathing sounds, etc.), impedance information, plethysmograph information, chemical information, temperature information, or other physiologic information of the patient.

To balance physiologic monitoring with power and resource usage of ambulatory medical devices, physiologic information can be detected in one or more detection windows, often between 30 seconds and 2 minutes, though in certain examples longer or shorter, such as between 15 seconds and 5 minutes, etc., occurring multiple times throughout a day (e.g., every hour, every 2 hours, daily, etc.). Ambulatory medical devices can determine information about each detection window or one or more detection windows, such as summary information or information representative of a respective detection window, daily information representative of information about the patient for a particular day, etc., and can additionally determine to store or transmit sensed or detected information, such as for transmission to a remote device, based on the determination. In certain examples, the ambulatory medical device can aggregate information from multiple sensors, detect various events using information from each sensor separately or in combination, update a detection status based on the information, and transmit a message or an alert to one or more remote devices that a detection has been made, that information has been stored or transmitted, such that one or more additional processes or systems can use the stored or transmitted detection or information for one or more other review or processes.

Ambulatory medical devices, such as short—or long-term insertable cardiac monitors (ICMs), etc., measure physiologic information over a period of time, for example, and can determine one or more patient conditions, including detected episodes of patient worsening, statuses, baselines, etc. Physiologic information associated with specific detection windows can be recorded, stored, and provided to a clinician. The number of detection windows and associated physiologic information recorded, transmitted, and reviewed by a clinician are often merely a subset of the total atrial fibrillation burden reported to the clinician or available for review (e.g., stored and not transmitted, transmitted and not reviewed, etc.). In many instances, more burden is reported than is reviewable (e.g., not recorded, stored, or transmitted, etc.).

Certain ambulatory medical devices have limitations, such as based on hardware limitations (e.g., power, processing resources, circuit components, etc.), on how many episodes or events can be recorded in a period of time (e.g., 5 detected episodes per day, etc.), how much physiologic information can be recorded in a single episode or event (e.g., 6 minutes per episode at a specific sampling frequency, etc.), or how much physiologic information can be transmitted in a period of time (e.g., 30 minutes per day at a specific sampling frequency, etc.), etc. Recorded and transmitted episodes can be reviewed and adjudicated (e.g., by a clinician, using one or more additional processes, or combinations thereof). However, incomplete physiologic information recorded and transmitted by such ambulatory medical devices having limitations (e.g., having ECG information from a portion of a determined episode) but not representative information from each detection window of the determined episode except that such detection window was determined to be an atrial fibrillation episode) may result in a greater number of false positive adjudications than actually occurred. Moreover, devices and algorithms can learn from adjudications, such that false positive adjudications can directly be fed back to ambulatory medical devices to conserve processing, storage, and transmission resources, further reducing the likelihood that false positive episodes will consume any limited resources, or reducing resources for operation, extending the lifespan or usable life of the ambulatory medical device.

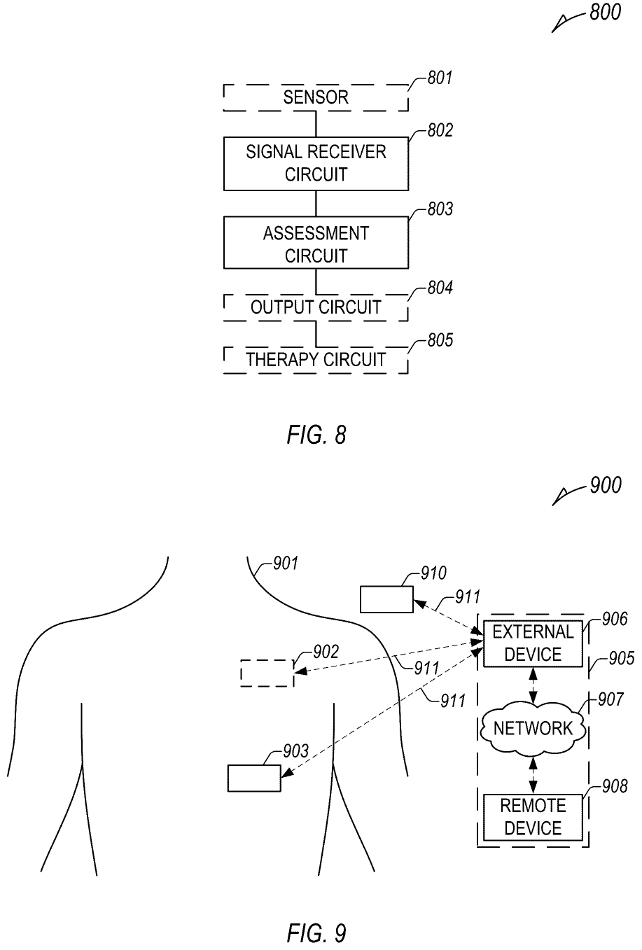

FIG. 8 illustrates an example system 800 (e.g., a medical device system). In an example, one or more aspects of the example system 800 can be a component of, or communicatively coupled to, a medical device, such as an implantable medical device (IMD), an insertable cardiac monitor (ICM), an ambulatory medical device (AMD), etc. The system 800 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 800 can include a single medical device or a plurality of medical devices implanted in a patient's body or otherwise positioned on or about the patient to monitor patient physiologic information of the patient using information from one or more sensors, such as a sensor 801. In an example, the sensor 801 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 800 can include a signal receiver circuit 802 and an assessment circuit 803. The signal receiver circuit 802 can be configured to receive physiologic information of a patient (or group of patients) from the sensor 801. The assessment circuit 803 can be configured to receive information from the signal receiver circuit 802, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 803 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 800 can include an output circuit 804 configured to provide an output to a user, or to cause an output to be provided to a user, such as through an output, a display, or one or more other user interface, the output including a score, a trend, an alert, or other indication. In other examples, the output circuit 804 can be configured to provide an output to another circuit, machine, or process, such as a therapy circuit 805 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 805 can include one or more of a stimulation control circuit, a cardiac stimulation circuit, a neural stimulation circuit, a dosage determination or control circuit, etc. In other examples, the therapy circuit 805 can be controlled by the assessment circuit 803, or one or more other circuits, etc. In certain examples, the assessment circuit 803 can include the output circuit 804 or can be configured to determine the output to be provided by the output circuit 804, while the output circuit 804 can provide the signals that cause the user interface to provide the output to the user based on the output determined by the assessment circuit 803.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 9 illustrates an example patient management system 900 and portions of an environment in which the patient management system 900 may operate. The patient management system 900 can perform a range of activities, including remote patient monitoring and diagnosis of a disease condition, programming of ambulatory medical devices, and control of one or more therapies. Such activities can be performed proximal to a patient 901, such as in a patient home or office, through a centralized server, such as in a hospital, clinic, or physician office, or through a remote workstation, such as a secure wireless mobile computing device.

The patient management system 900 can include one or more medical devices, an external system 905, and a communication link 911 providing for communication between the one or more ambulatory medical devices and the external system 905. The one or more medical devices can include an ambulatory medical device (AMD), such as an implantable medical device (IMD) 902, a wearable medical device 903, or one or more other implantable, leadless, subcutaneous, external, wearable, or medical devices configured to monitor, sense, or detect information from, determine physiologic information about, or provide one or more therapies to treat various conditions of the patient 901, such as one or more cardiac or non-cardiac conditions (e.g., dehydration, sleep disordered breathing, etc.).

In an example, the implantable medical device 902 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 901. In another example, the implantable medical device 902 can include a monitor implanted, for example, subcutaneously in the chest of patient 901, the implantable medical device 902 including a housing containing circuitry and, in certain examples, one or more sensors, such as a temperature sensor, etc.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICD), subcutaneous implantable cardioverter defibrillators, cardiac resynchronization therapy defibrillators (CRT-Ds), insertable cardiac monitors, leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 902 can include a signal receiver circuit or an assessment circuit configured to detect or determine specific physiologic information of the patient 901, or to determine one or more conditions or provide information or an alert to a user, such as the patient 901 (e.g., a patient), a clinician, or one or more other caregivers or processes, such as described herein. The implantable medical device 902 can alternatively or additionally be configured as a therapeutic device configured to treat one or more medical conditions of the patient 901. The therapy can be delivered to the patient 901 via the lead system and associated electrodes or using one or more other delivery mechanisms. The therapy can include delivery of one or more drugs to the patient 901, such as using the implantable medical device 902 or one or more of the other ambulatory medical devices, etc. In some examples, therapy can include cardiac resynchronization therapy for rectifying dyssynchrony and improving cardiac function in heart failure patients. In other examples, the implantable medical device 902 can include a drug delivery system, such as a drug infusion pump to deliver drugs to the patient for managing arrhythmias or complications from arrhythmias, hypertension, hypotension, or one or more other physiologic conditions. In other examples, the implantable medical device 902 can include one or more electrodes configured to stimulate the nervous system of the patient or to provide stimulation to the muscles of the patient airway, etc.

The wearable medical device 903 can include one or more wearable or external medical sensors or devices (e.g., automatic external defibrillators (AEDs), Holter monitors, patch-based devices, smart watches, smart accessories, wrist—or finger-worn medical devices, such as a finger-based photoplethysmography sensor, etc.).

The external system 905 can include a dedicated hardware/software system, such as a programmer, a remote server-based patient management system, or alternatively a system defined predominantly by software running on a standard personal computer. The external system 905 can manage the patient 901 through the implantable medical device 902 or one or more other ambulatory medical devices connected to the external system 905 via a communication link 911. In other examples, the implantable medical device 902 can be connected to the wearable medical device 903, or the wearable medical device 903 can be connected to the external system 905, via the communication link 911. This can include, for example, programming or reprogramming the implantable medical device 902 with different parameter settings to perform one or more of acquiring physiologic data, performing at least one self-diagnostic test (such as for a device operational status), analyzing the physiologic data, or optionally delivering or adjusting a therapy for the patient 901. Additionally, the external system 905 can send information to, or receive information from, the implantable medical device 902 or the wearable medical device 903 via the communication link 911. Examples of the information can include real-time or stored physiologic data from the patient 901, diagnostic data, such as detection of patient hydration status, hospitalizations, responses to therapies delivered to the patient 901, or device operational status of the implantable medical device 902 or the wearable medical device 903 (e.g., battery status, lead impedance, etc.). The communication link 911 can be an inductive telemetry link, a capacitive telemetry link, or a radio-frequency (RF) telemetry link, such as a wireless telemetry based on, for example, “strong” Bluetooth® or IEEE 802.11 wireless fidelity “Wi-Fi” interfacing standards. Other configurations and combinations of patient data source interfacing are possible.

The external system 905 can include an external device 906 in proximity of the one or more ambulatory medical devices, and a remote device 908 in a location relatively distant from the one or more ambulatory medical devices, in communication with the external device 906 via a communication network 907. Examples of the external device 906 can include a medical device programmer. The remote device 908 can be configured to evaluate collected patient or patient information and provide alert notifications, among other possible functions. In an example, the remote device 908 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 908 can receive data from multiple patients. The data can be collected by the one or more ambulatory medical devices, among other data acquisition sensors or devices associated with the patient 901. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 905 or one or more components thereof (e.g., the external device 906, the remote device 908, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 908 may additionally include one or more locally configured clients or remote clients securely connected over the communication network 907 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 908, including the server and the interconnected clients, may also execute a follow-up scheme by sending follow-up requests to the one or more ambulatory medical devices, or by sending a message or other communication to the patient 901 (e.g., the patient), clinician or authorized third party as a compliance notification.

The communication network 907 can provide wired or wireless interconnectivity. In an example, the communication network 907 can be based on the Transmission Control Protocol/Internet Protocol (TCP/IP) network communication specification, although other types or combinations of networking implementations are possible. Similarly, other network topologies and arrangements are possible.

One or more of the external device 906 or the remote device 908 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 906 or the remote device 908 can include a respective display unit for displaying the physiologic or functional signals, or alerts, alarms, emergency calls, or other forms of warnings to signal the detection of one or more conditions. In some examples, the external system 905 can a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 902, the wearable medical device 903, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 908, or via a user interface of a software application executing on a client device communicatively connected with the remote device 908. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 905 can be implemented using hardware, software, firmware, or combinations thereof. Portions of the one or more ambulatory medical devices or the external system 905 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 910 can be configured to send information to or receive information from one or more of the ambulatory medical devices or the external system 905 using the communication link 911. In an example, the one or more ambulatory medical devices, the external device 906, or the remote device 908 can be configured to control one or more parameters of the therapy device 910. The external system 905 can allow for programming or reprogramming the one or more ambulatory medical devices and can receive information about one or more signals acquired by the one or more ambulatory medical devices, such as can be received via a communication link 911. The external system 905 can include a local external implantable medical device programmer. The external system 905 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows, 4-minute windows, etc.) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, and to disrupt or reduce the impact of the detected atrial fibrillation event, or a combination thereof.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

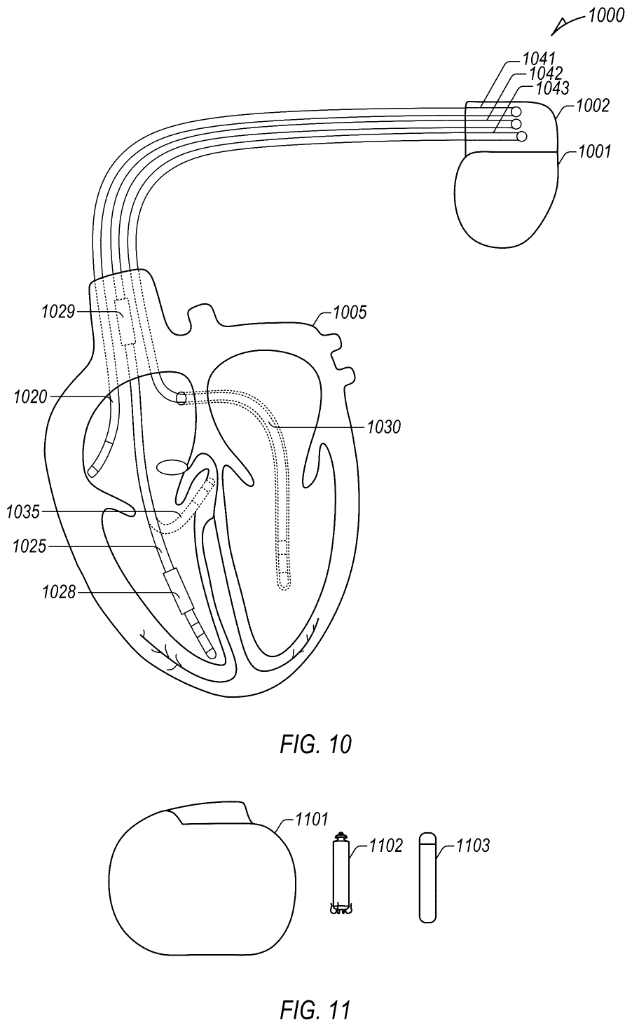

FIG. 10 illustrates an example implantable medical device system 1000 including an implantable medical device 1001 electrically coupled to a heart 1005, such as through one or more leads coupled to the implantable medical device 1001 through one or more lead ports, including first, second, or third lead ports 1041, 1042, 1043 in a header 1002 of the implantable medical device 1001. In an example, the implantable medical device 1001 can include an antenna, such as in the header 1002, configured to enable communication with an external system and one or more electronic circuits (e.g., an assessment circuit, etc.) in a hermetically sealed housing (CAN).

The implantable medical device 1001 may include an insertable cardiac monitor, pacemaker, defibrillator, cardiac resynchronization therapy device, or other subcutaneous implantable medical device or cardiac rhythm management device configured to be implanted in a chest of a patient, having one or more leads to position one or more electrodes or other sensors at various locations in or near the heart 1005, such as in one or more of the atria or ventricles. Separate from, or in addition to, the one or more electrodes or other sensors of the leads, the implantable medical device system 1000 can include one or more electrodes or other sensors (e.g., a pressure sensor, an accelerometer, a gyroscope, a microphone, etc.) powered by a power source in the implantable medical device 1001. The one or more electrodes or other sensors of the leads, the implantable medical device 1001, or a combination thereof, can be configured to detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 1005 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 1001 can include one or more electronic circuits configured to sense one or more physiologic signals, such as an electrogram or a signal representing mechanical function of the heart 1005. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 1028, the second defibrillation coil electrode 1029, etc.) may be used together with the housing to deliver one or more cardioversion/defibrillation pulses.

In an example, the implantable medical device 1001 can sense impedance such as between electrodes located on one or more of the leads or the housing. The implantable medical device 1001 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 1001 can be configured to inject current between an electrode on one or more of the first, second, third, or fourth leads 1020, 1025, 1030, 1035 and the housing, and to sense the resultant voltage between the same or different electrodes and the housing.

The implantable medical device 1001 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, right ventricular pressure, left ventricular coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are contemplated.

FIG. 11 illustrates example implantable medical devices, including a subcutaneous implantable cardioverter defibrillator 1101, a leadless cardiac pacemaker 1102 including tines for placement, and an insertable cardiac monitor 1103.

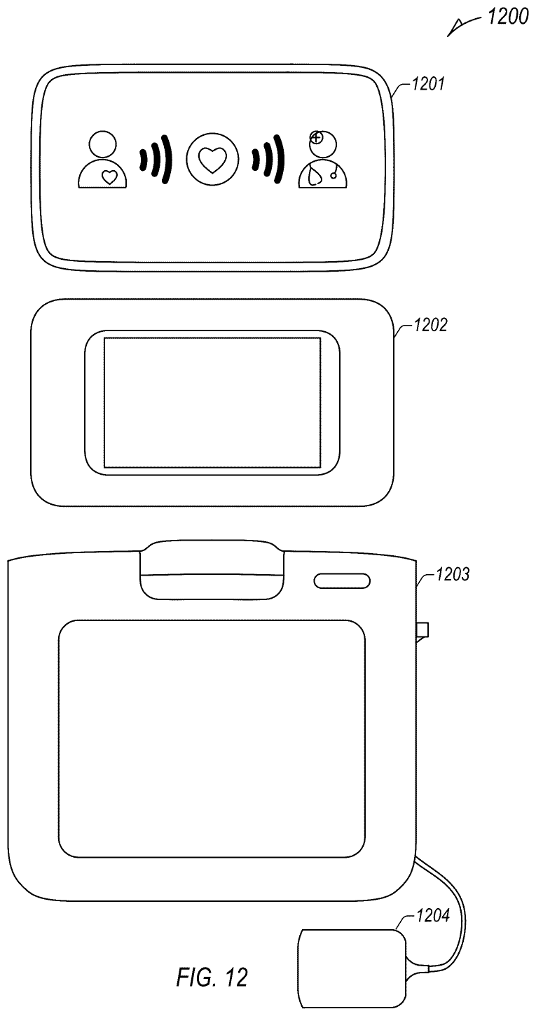

FIG. 12 illustrates example remote patient management systems 1200 for communication with an ambulatory medical device, such as for receiving information from or providing information to, including programming instructions, one or more ambulatory medical devices. The example remote patient management systems 1200 include a first remote patient management device 1201 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 1202 (e.g., an EMBLEM™ S-ICD Programmer) with RF telemetry capabilities through one or more communication circuits and an optional external telemetry wand for communication with an ambulatory medical device, and a third remote patient management system 1203 (e.g., a LATITUDE™ Programming System, Model 3300, etc.) with RF telemetry capabilities through one or more communication circuits and an external telemetry wand 1204 (e.g., a Model 6395 Telemetry Wand, etc.) including an external telemetry coil configured for inductive communication with a corresponding telemetry coil of an implantable medical device. Although not illustrated herein, the remote patient management systems 1200 can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

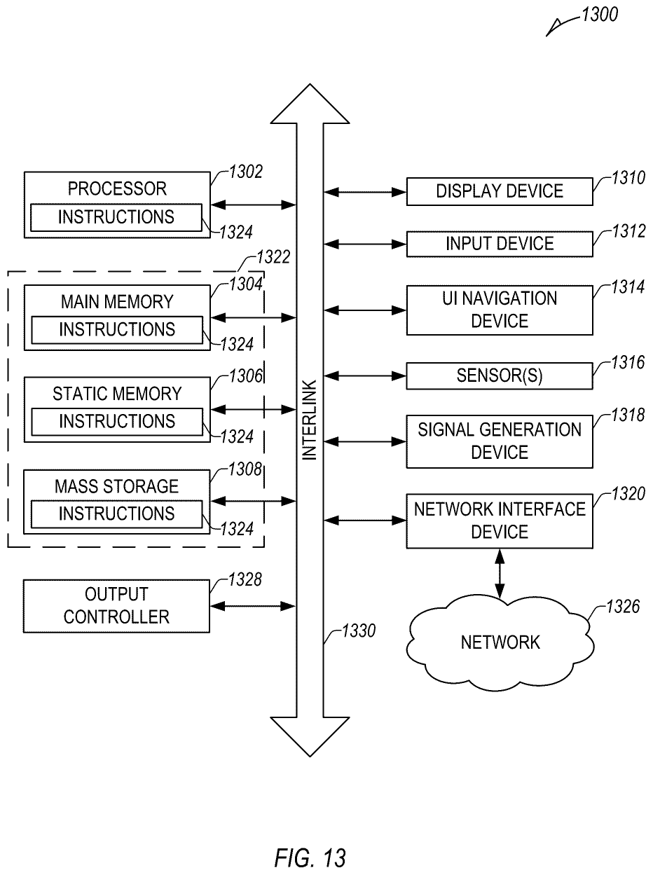

FIG. 13 illustrates a block diagram of an example machine 1300 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1300. Circuitry (e.g., processing circuitry, an assessment circuit, etc.) is a collection of circuits implemented in tangible entities of the machine 1300 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1300 follow.

In alternative embodiments, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1300 (e.g., computer system) may include a hardware processor 1302 (e.g., a central processing unit (CPU), a graphics processing unit (GPU), a hardware processor core, or any combination thereof), a main memory 1304, a static memory 1306 (e.g., memory or storage for firmware, microcode, a basic-input-output (BIOS), unified extensible firmware interface (UEFI), etc.), and mass storage 1308 (e.g., hard drive, tape drive, flash storage, or other block devices) some or all of which may communicate with each other via an interlink 1330 (e.g., bus). The machine 1300 may further include a display unit 1310, an input device 1312 (e.g., a keyboard), and a user interface (UI) navigation device 1314 (e.g., a mouse). In an example, the display unit 1310, input device 1312, and UI navigation device 1314 may be a touch screen display. The machine 1300 may additionally include a signal generation device 1318 (e.g., a speaker), a network interface device 1320, and one or more sensors 1316, such as a global positioning system (GPS) sensor, compass, accelerometer, or one or more other sensors. The machine 1300 may include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 1302, the main memory 1304, the static memory 1306, or the mass storage 1308 may be, or include, a machine-readable medium 1322 on which is stored one or more sets of data structures or instructions 1324 (e.g., software) embodying or utilized by any one or more of the techniques or functions described herein. The instructions 1324 may also reside, completely or at least partially, within any of registers of the hardware processor 1302, the main memory 1304, the static memory 1306, or the mass storage 1308 during execution thereof by the machine 1300. In an example, one or any combination of the hardware processor 1302, the main memory 1304, the static memory 1306, or the mass storage 1308 may constitute the machine-readable medium 1322. While the machine-readable medium 1322 is illustrated as a single medium, the term “machine-readable medium” may include a single medium or multiple media (e.g., a centralized or distributed database, and/or associated caches and servers) configured to store the one or more instructions 1324.

The term “machine-readable medium” may include any medium that is capable of storing, encoding, or carrying instructions for execution by the machine 1300 and that cause the machine 1300 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1324 may be further transmitted or received over a communications network 1326 using a transmission medium via the network interface device 1320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communications network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1300, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system for improving heart failure readmission risk determination to optimize resources of the medical device system, comprising:
a signal receiver circuit configured to receive physiologic information of a patient; and
an assessment circuit configured to:
analyze the physiologic information occurring over specific time periods relative to a prior heart failure event with respect to a baseline value for the patient;
adjust, in response to a detected or received trigger event, the baseline value for the patient from a long-term baseline value to one of a shorter-term baseline value or a static value indicative of a baseline value at a prior time relative to the detected or received trigger event;
determine a readmission score for the patient based on the analyzed physiologic information, the readmission score for the patient indicative of a risk of subsequent readmission after treatment or discharge from hospitalization or treatment of the prior heart failure event; and
provide a control signal to control a mode or operation of the medical device system based on the readmission score to optimize resources of the medical device system.

2. The medical device system of claim 1, wherein the assessment circuit is configured to determine the readmission score with respect to the adjusted baseline value for the patient.

3. The medical device system of claim 1, wherein the assessment circuit is configured to provide the control signal based on the readmission score in response to the determined readmission score exceeding the adjusted baseline value by a threshold.

4. The medical device system of claim 1, wherein the assessment circuit is configured to determine the readmission score for the patient as a composite function of the analyzed physiologic information, including two or more of: S1 heart sound information, S3 heart sound information, thoracic impedance, activity information, respiration information, and heart rate.

5. The medical device system of claim 4, wherein the signal receiver circuit or the assessment circuit is configured to receive an indication of patient discharge from hospitalization or treatment of the prior heart failure event,
wherein to determine the readmission score for the patient includes to determine an indication that the patient is likely to experience readmission or an indication that the prior heart failure event and discharge is likely to result in readmission within a specific time period after the received indication of patient discharge from hospitalization or treatment of the prior heart failure event.

6. The medical device system of claim 1, wherein the readmission score is indicative of a future heart failure event.

7. The medical device system of claim 1, wherein the assessment circuit is configured to determine the baseline value for the patient, including to:
determine the long-term baseline value as a function of 30 or more days of preceding physiologic information of the patient; and
determine the shorter-term baseline value as a function of a time period of physiologic information of the patient between 3 and 10 days of preceding physiologic information of the patient,
wherein to adjust the baseline value for the patient includes to adjust the baseline value for the patient from the long-term baseline to the shorter-term baseline value.

8. The medical device system of claim 1, wherein the assessment circuit is configured to determine the baseline value for the patient, including to:
determine the long-term baseline value as a function of 30 or more days of preceding physiologic information of the patient; and
determine the static value indicative of the baseline value at a prior time relative to the detected or received trigger event, the prior time including at least 30 days prior to the detected or received trigger event,
wherein to adjust the baseline value for the patient includes to adjust the baseline value for the patient from the long-term baseline to the static value.

9. The medical device system of claim 1, wherein the assessment circuit is configured to determine the baseline value as a function of the long-term baseline value, the shorter-term baseline value, and the static value over a transition period until the determined readmission score or the long-term baseline meets the static value.

10. The medical device system of claim 1, wherein the assessment circuit is configured to analyze S3 heart sound information over first and second time periods and to determine the readmission score for the patient as function of a difference in the S3 heart sound information over the first and second time periods, wherein the first time period is before the prior heart failure event and the second time period is after discharge from hospitalization or treatment of the prior heart failure event.

11. The medical device system of claim 10, wherein the first time period is between −25 days and −5 days before the prior heart failure event and the second time period is between 0 days and 30 days after discharge from hospitalization or treatment of the prior heart failure event.

12. The medical device system of claim 1, wherein the assessment circuit is configured to determine the readmission score as a function of:
a first composite function of the analyzed physiologic information over a first time period before the prior heart failure event, the physiologic information including two or more of: S1 heart sound information, S3 heart sound information, thoracic impedance, activity information, respiration information, and heart rate information;
a second composite function of S3 heart sound information and S1 heart sound information over a time period before the prior heart failure event; and
an integrated value of the first composite function over a second time period after discharge from hospitalization or treatment of the prior heart failure event.

13. The medical device system of claim 1, wherein the trigger event is one or more of a received request to adjust the respective baseline value, a detected in-alert state, or a received or detected indication of a first heart failure event or hospitalization or treatment of the first heart failure event of the patient,
wherein the first heart failure event is the prior heart failure event.

14. A method for improving heart failure readmission risk determination to optimize resources of a medical device system, comprising:
receiving, using a signal receiver circuit, physiologic information of a patient;
analyzing, using an assessment circuit, the physiologic information occurring over specific time periods relative to a prior heart failure event with respect to a baseline value for the patient;
adjusting, using the assessment circuit, in response to a detected or received trigger event, the baseline value for the patient from a long-term baseline value to one of a shorter-term baseline value or a static value indicative of a baseline value at a prior time relative to the detected or received trigger event;
determining, using the assessment circuit, a readmission score for the patient based on the analyzed physiologic information, the readmission score for the patient indicative of a risk of subsequent readmission after treatment or discharge from hospitalization or treatment of the prior heart failure event; and
providing, using the assessment circuit, a control signal to control a mode or operation of the medical device system based on the readmission score to optimize resources of the medical device system.

15. The method of claim 14, wherein providing the control signal based on the readmission score includes providing the control signal in response to the determined readmission score exceeding the adjusted baseline value.

16. The method of claim 14, wherein determining the readmission score for the patient includes determining a composite function of the analyzed physiologic information, including two or more of: S1 heart sound information, S3 heart sound information, thoracic impedance, activity information, respiration information, and heart rate.

17. The method of claim 16, comprising:
receiving an indication of patient discharge from hospitalization or treatment of the prior heart failure event,
wherein determining the readmission score for the patient includes determining an indication that the patient is likely to experience readmission or an indication that the prior heart failure event and discharge is likely to result in readmission within a specific time period after the received indication of patient discharge from hospitalization or treatment of the prior heart failure event.

18. The method of claim 14, comprising:
determining the baseline value for the patient, including:
determining the long-term baseline value as a function of 30 or more days of preceding physiologic information of the patient; and
determining the shorter-term baseline value as a function of a time period of physiologic information of the patient between 3 and 10 days of preceding physiologic information of the patient,
wherein adjusting the baseline value for the patient includes adjusting the baseline value for the patient from the long-term baseline to the shorter-term baseline value.

19. The method of claim 14, comprising:
determining the baseline value for the patient, including:
determining the long-term baseline value as a function of 30 or more days of preceding physiologic information of the patient; and
determine the static value indicative of the baseline value at a prior time relative to the detected or received trigger event, the prior time including at least 30 days prior to the detected or received trigger event,
wherein adjusting the baseline value for the patient includes adjusting the baseline value for the patient from the long-term baseline to the static value.

20. The method of claim 14, wherein analyzing the physiologic information comprises analyzing S3 heart sound information over first and second time periods,
wherein determining the readmission score for the patient includes as function of a difference in the S3 heart sound information over the first and second time periods, wherein the first time period is before the prior heart failure event and the second time period is after discharge from hospitalization or treatment of the prior heart failure event,
wherein the first time period is between −25 days and −5 days before the prior heart failure event and the second time period is between 0 days and 30 days after discharge from hospitalization or treatment of the prior heart failure event.
